SEQUENCE LISTING (1 sequence)

SEQ ID NO: 1 - protein, 572 aa, Homo sapiens
MSYQGKKNIPRITSDRLLIKGGKIVNDDQSFYADIYMEDGLIKQIGENLIVPGGVKTIEAHSRMVIPGGIDVHTRFQMPDQGMTSADDFFQGTKAALAGGTTMIIDHVVPEPGTSLLAAFDQWREWADSKSCCDYSLHVDISEWHKGIQEEMEALVKDHGVNSFLVYMAFKDRFQLTDCQIYEVLSVIRDIGAIAQVHAENGDIIAEEQQRILDLGITGPEGHVLSRPEEVEAEAVNRAITIANQTNCPLYITKVMSKSSAEVIAQARKKGTVVYGEPITASLGTDGSHYWSKNWAKAAAFVTSPPLSPDPTTPDFLNSLLSCGDLQVTGSAHCTFNTAQKAVGKDNFTLIPEGTNGTEERMSVIWDKAVVTGKMDENQFVAVTSTNAAKVFNLYPRKGRIAVGSDADLVIWDPDSVKTISAKTHNSSLEYNIFEGMECRGSPLVVISQGKIVLEDGTLHVTEGSGRYIPRKPFPDFVYKRIKARSRLAELRGVPRGLYDGPVCEVSVTPKTVTPASSAKTSPAKQQAPPVRNLHQSGFSLSGAQIDDNIPRRTTQRIVAPPGGRANITSLG

(19) United States
(12) Patent Application Publication
(10) Pub. No.: US 2012/0135009 A1
(43) Pub. Date: May 31, 2012

(54) Cleaved and Phosphorylated CRMP2 as Blood Marker of Inflammatory Diseases of the Central Nervous System

(76) Inventor: Pascale Giraudon, Lyon (FR)

(21) Appl. No.: 13/383,678

(22) PCT Filed: Jul. 16, 2009
(86) PCT No.: PCT/IB2009/054212

§ 371 Date: Feb. 6, 2012

(51) Int. Cl. A61K 39/395; C07K 16/18; A61P 25/28; A61P 29/00; A61P 25/00; A61P 31/04; A61P 31/12; G01N 33/53; A61P 25/16

(52) U.S. Cl. 424/172.1; 435/7.1; 436/501; 530/389.1

(57) ABSTRACT

The present invention relates to a method for in vitro prognosis, diagnosis and/or monitoring of an inflammatory disease of the central nervous system in a subject, said method comprising detecting, in a sample of cells of the immune system from the subject, the presence of a Collapsin Response Mediator Protein 2 (CRMP2) which is phosphorylated on tyrosine 479 (Y479), and optionally further phosphorylated on serine 465 (S465), wherein the detection of the presence of Y479-phosphorylated CRMP2, which is optionally further phosphorylated on serine 465, is indicative of an inflammatory disease of the central nervous system.

### FIELD OF THE INVENTION

The present invention relates to methods for predicting, diagnosing and/or treating inflammatory diseases of the central nervous system.

### BACKGROUND OF THE INVENTION

Neuroinflammatory diseases, or inflammatory diseases of the central nervous system are characterized by abrupt neurologic deficits associated with inflammation, and usually demyelination, and axonal damage. In these disorders, neuroinflammation damages the myelin sheath that insulates nerve cell fibers in the brain and spinal cord, which causes extensive and often permanent damage to the underlying nerves. Patients suffering from a neuroinflammatory disease experience dramatic and sometimes permanent losses in sensory and motor function. Due to the prevalence, morbidity, and mortality associated with neuroinflammatory diseases, they represent a significant medical, social, and financial burden. It is estimated that these neurological conditions affect more than five million people in North America and generate costs of care that exceed US$ 75 billion annually.

Neuroinflammatory diseases are difficult to diagnose and treat. Unfortunately inaccurate diagnoses result in uncertainty for patients. Quick and accurate methods of diagnosing neuroinflammatory diseases are thus important to ensure that appropriate methods of treatment are implemented to ameliorate neuroinflammatory symptoms and preserve neurological function. Accordingly, there is a need for new methods for predicting, diagnosing and/or treating inflammatory diseases of the central nervous system.

The present inventors had shown, in the international application WO2003/022298, the potential use of the Collapsin Response Mediator Protein 2 (CRMP2) for the treatment, prognosis or diagnosis of pathologies related to a dysfunction of the immune system. More specifically, they had shown that CRMP2 was present at high levels in the T lymphocytes in patients affected with dysimmune pathologies, and that the nuclear translocation of a highly phosphorylated form of CRMP2 was increased in lymphocytes infected with HTLV-1 or T lymphocytes of patients having an immune deficiency related to the Fas/Fas ligand system.

CRMPs are a family of 5 members which are known to be modulators of the cytoskeleton rearrangement during the axonal growth in the central nervous system (CNS). In T lymphocytes and in the CNS, CRMP2 both displays a 62 kDa full-length form (CRMP2-62) and a 58 kDa cleaved form (CRMP2-58). Four phosphorylated forms of CRMP2 have been described so far: CRMP2 phosphorylated on serine 522 (pCRMP2-Ser522), CRMP2 phosphorylated on threonine 509 and 514 (pCRMP2-Thr509/514), CRMP2 phosphorylated on threonine 555 (pCRMP2-Thr555) and CRMP2 phosphorylated on serine 465 (Uchida et al. (2005) Genes Cells 10:165-179; Cole et al. (2006) J Biol Chem 281:16581-16588).

The present inventors have identified a new site of phosphorylation of CRMP2: tyrosine 479 (Y479). They have demonstrated that Y479 phosphorylation was induced by the activation of the membrane CXCR4 receptor of T lymphocytes by the CXCL12 chemokine (Varrin-Doyer et al. (2009) J Biol Chem 284:13265-13276), whereas S465 phosphorylation was induced after the T cell receptor (TCR) stimulation.

### SUMMARY OF THE INVENTION

The present invention arises from the unexpected finding, by the inventors, (i) that Y479 mutation decreases the T cell migration capacity including T cell polarization and T cell migratory rate, which shows the importance of Y479-phosphorylated CRMP2 in the neuroinflammatory process, and (ii) that patients suffering from multiple sclerosis or from myelopathy induced by HTLV-1 displayed a population of activated T cells with a high level of Ser465-phosphorylated cleaved CRMP2. These phosphorylations and cleavage have the advantage to be easily detectable by Western Blot or flow cytometry in immune cells of patients.

Thus, the present invention relates to a method for in vitro prognosis, diagnosis and/or monitoring of an inflammatory disease of the central nervous system in a subject, said method comprising detecting, in a sample of cells of the immune system from the subject, the presence of a Collapsin Response Mediator Protein 2 (CRMP2) which is phosphorylated on tyrosine 479 (Y479), wherein the detection of the presence of Y479-phosphorylated CRMP2 is indicative of an inflammatory disease of the central nervous system.

The present invention also relates to an antibody specific of a CRMP2 which is phosphorylated on tyrosine 479, and to its use in the prognosis, diagnosis and/or monitoring of an inflammatory disease of the central nervous system, to its use for decreasing immune cells migration, and to its use in the treatment of an inflammatory disease of the central nervous system.

The present invention also relates to an antibody as defined above for detecting a CRMP2 which is phosphorylated on tyrosine 479 and/or on serine 465.

The present invention also relates to an antagonist of the CXCR4 receptor for use for decreasing T lymphocytes migration, and for use in the treatment of an inflammatory disease of the central nervous system.

### DETAILED DESCRIPTION OF THE INVENTION

### Inflammatory Diseases of the Central Nervous System

As used herein, an “inflammatory disease of the central nervous system” or “neuroinflammatory disease” denotes a disease of the central nervous system associated with inflammation, demyelination, or axonal and/or neuronal damage. Inflammatory diseases of the central nervous system (CNS) can be non-infectious or infectious. Non-infectious diseases that can cause inflammatory lesions include some toxins, autoimmune diseases and immune-mediated conditions. Viruses, bacteria, fungi, protozoa and metazoan parasites all can cause inflammatory diseases of the CNS. Inflammatory diseases of the CNS are in particular gathered in codes G00 to G09 of the International Statistical Classification of Diseases and Related Health Problems published by the WHO. Examples of inflammatory diseases of the CNS include viral, bacterial or parasitic infections with meningitis, encephalitis, myelitis, myelopathy or encephalomyelitis; intracranial and intrathecal abcess and granuloma; intracranial and intrathecal phlebitis and thrombophlebitis; multiple sclerosis; Alzheimer disease and Parkinson disease. Preferably, the inflammatory disease of the CNS according to the invention is selected from the group consisting of viral or bacterial infections with meningitis, encephalitis, myelitis, encephalomyelitis, encephalititis or myelopathy, multiple sclerosis, Parkinson disease and Alzheimer disease. More preferably, the inflammatory disease of the CNS according to the invention is selected from the group consisting of viral infection with encephalitis, multiple sclerosis, Alzheimer disease and Parkinson disease.

As used herein, “viral infections with meningitis, encephalitis, myelitis, myelopathy or encephalomyelitis” include viral infections due to retroviruses, adenoviruses, enteroviruses, herpesvirus, measles virus, mumps virus, rubella virus, smallpox virus, chickenpox, varicella-zoster virus, influenza virus, cytomegalovirus, poliovirus, HTLV-1 and Epstein-Barr virus. Preferably, viral infections according to the invention are selected from the group consisting of viral infections due to HTLV-1 or HIV.

As used herein, “bacterial infections with meningitis, encephalitis, myelitis, myelopathy or encephalomyelitis” include bacterial infections due to Haemophilis influenzae, Streptococcus pneumoniae, Streptococcus pyogenes, Streptococcus agalactiae, Staphylococcus aureus, Neisseria meningitidis, Escherichia coli, Klebsiella, Friedländer bacillus, Bacillus anthracis, Neisseria gonorrhoeae, Leptospira, Listeria monocytogenes, Borrelia, Treponema pallidum, Salmonella, Mycobacterium tuberculosis and Salmonella enterica.

As used herein, “parasitic infections with meningitis, encephalitis, myelitis, myelopathy or encephalomyelitis” include parasitic infections due to Trypanosoma, in particular Trypanosoma brucei and Trypanosoma cruzi, Toxoplasma gondii, and Naegleria fowleri.

### CRMP2

As used herein, the “Collapsin Response Mediator Protein 2” or “CRMP2” or “ULIP2” refers to a phosphoprotein, first described in neuron growth cone advance (Goshima et al. (1995) Nature 376:509-514; Charrier et al. (2003) Mol. Neurobiol. 28:51-64) and neural cell migration via microtubule organization. It is a member of the CRMP/TOAD/Ulip/DRP family of cytosolic phosphoproteins. Preferably, CRMP2 comprises, or consists in, the amino acid sequence SEQ ID NO: 1.

In the context of the invention, the inventors have demonstrated that CRMP2 could be phosphorylated on tyrosine 479 (Y479). Additionally, Y479-phosphorylated CRMP2 may be either in a full-length form or in a cleaved form. The full-length form of Y479-phosphorylated CRMP2 corresponds to the full-length form of 62 kDa of CRMP2 (CRMP2-62), while the cleaved form corresponds to the cleaved form of 58 kDa of CRMP2 (CRMP2-58). More particularly, CRMP2-58 is obtained by cleavage of CRMP2-62 at the cleavage site described in Rogemond et al. situated between amino acids 489 and 532 of CRMP2 (Rogemond et al. (2008) J. Biol. Chem. 283:14751-14761). CRMP2 and Y479-phosphorylated CRMP2, in particular in their cleaved form, may be further phosphorylated on serine 465 (S465).

### Antibodies and Uses Thereof

The present invention relates to an antibody specific of a CRMP2 which is phosphorylated on tyrosine 479.

As used herein, the term “antibody” refers to immunoglobulin molecules and immunologically active portions of these immunoglobulin molecules, i.e. molecules which contain an antigen binding site which specifically binds an antigen. The term “antibody” thus does not only include whole antibody molecules but also antibody fragments as well as variants (including derivatives) of antibodies and of antibody fragments. An antibody according to the invention may be a polyclonal or a monoclonal antibody.

As used herein, a “monoclonal antibody” refers to an antibody of a single amino acid composition, that is directed against a specific antigen and that may be recombinant or produced for example by a single clone of B cells or hybridoma.

“Antibody fragments” comprise a portion of an intact antibody, preferably the variable region or the antigen binding region of an intact antibody. Examples of suitable antibody fragments include Fv, Fab, Fab′, (Fab′)₂, Fd, dAb, scFV, dsFV, sc(Fv)₂ fragments and diabodies. The antibody according to the invention may also be a camelid nanobody. The antibody according to the invention may be a modified antibody. In particular, the antibody according to the invention may be conjugated to a marker moiety. The marker moiety may be for example a non-radioactive marker moiety such as a fluorophore, a coenzyme such as biotin, proteins, peptides, carbohydrates, lipids, dyes, polyethylene glycol, and the like.

The term “specific”, when referring to recognition or binding of a ligand to a target, means that the ligand interacts with the target without substantial interaction with another target that does display any structural similarity with the target. In particular, the antibody specific of Y479-phosphorylated CRMP2 as defined above specifically recognizes and binds to CRMP2 when CRMP2 has a phosphate group on tyrosine residue 479, but not when CRMP2 does not have a phosphate group on tyrosine residue 479. Preferably, the antibody specific of Y479-phosphorylated CRMP2 as defined above also recognizes and binds to CRMP2 when CRMP2 further has a phosphate group on serine 465.

The present invention also relates to an antibody specific of a CRMP2 which is phosphorylated on serine 465 (S465-phosphorylated CRMP2). In particular, said antibody specific of S465-phosphorylated CRMP2 as defined above specifically recognizes and binds to CRMP2 when CRMP2 has a phosphate group on serine residue 465, but not when CRMP2 does not have a phosphate group on tyrosine residue 465. Preferably, the antibody specific of S465-phosphorylated CRMP2 as defined above also recognizes and binds to CRMP2 when CRMP2 further has a phosphate group on tyrosine 479.

The antibodies as defined above recognize and bind to full-length and/or cleaved phosphorylated CRMP2. Preferably, the antibody specific of S465-phosphorylated CRMP2 as defined above recognizes and binds to cleaved S465-phosphorylated CRMP2.

Methods for producing polyclonal and monoclonal antibodies that react specifically with an antigen of interest are known to those of skill in the art (see, e.g., Coligan (1991) Current Protocols in Immunology Wiley/Greene, NY; Harlow and Lane (1989) Antibodies: A Laboratory Manual Cold Spring Harbor Press, NY; Stites et al. (eds.); Basic and Clinical Immunology (4^{th} ed.) Lange Medical Publications, Los Altos, Calif.: Goding (1986) Monoclonal Antibodies: Principles and Practice (2^{d} ed.) Academic Press, New York, N.Y.; and Kohler and Milstein (1975) Nature 256:495-497).

In order to produce antisera containing antibodies according to the invention with the desired specificity, the phosphorylated CRMP2 can be used to immunize suitable animals, e.g., mice, rabbits, or primates. A standard adjuvant, such as Freund's adjuvant, can be used in accordance with a standard immunization protocol. The animal's immune response to the immunogen preparation may be monitored by taking test bleeds and determining the titer of reactivity to the antigen of interest. Further fractionation of the antisera to enrich antibodies specifically reactive to the antigen and purification of the antibodies can be accomplished subsequently, using methods well known from those skilled in the art.

Monoclonal antibodies may be obtained using various techniques familiar to those of skill in the art. Typically, spleen cells from an animal immunized with a desired antigen are immortalized, commonly by fusion with a myeloma cell (Kohler and Milstein (1976) Eur. J. Immunol. 6:511-519). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and the yield of the monoclonal antibodies produced by such cells may be enhanced by various techniques, including injection into the peritoneal cavity of a vertebrate host. Additionally, monoclonal antibodies may also be recombinantly produced upon identification of nucleic acid sequences encoding an antibody with desired specificity or a binding fragment of such antibody by screening a human B cell cDNA library according to the general protocol outlined by Huse et al. (1989) Science 246:1275-1281. A monoclonal antibody may also be produced using recombinant DNA methods (see, e.g., U.S. Pat. No. 4,816,567) or by phage-display. The term “phage display” refers herein to a method for selecting ligands expressed on a bacteriophage capsid and encoded by a nucleic sequence inserted in the capsid encoding gene. This method is well known from those skilled in the art and is especially described by Scott and Smith (1990) Science 249:386-390, and Marks et al. (1991) J. Mol. Biol. 222:581-597.

In a particular embodiment, the above defined antibodies are humanized monoclonal antibodies. A “humanized antibody” refers to a non-human antibody that has been modified so that it more closely matches (in amino acid sequence) a human antibody. In certain embodiments, amino acid residues outside of the antigen binding residues of the variable region of the non-human antibody are modified. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In certain embodiments, a humanized antibody is constructed by replacing all or a portion of a CDR of a human antibody with all or a portion of a CDR from another antibody, such as a non-human antibody, having the desired antigen binding specificity. In certain embodiments, a humanized antibody comprises variable regions in which all or substantially all of the CDRs correspond to CDRs of a non-human antibody and all or substantially all of the framework regions (FRs) correspond to FRs of a human antibody. In certain such embodiments, a humanized antibody further comprises a constant region (Fc) of a human antibody. Furthermore, humanized antibodies may comprise residues that are not found in the recipient antibody or in the donor antibody. These modifications are made to further refine antibody performance. For further details, see Jones et al. (1986) Nature 321:522-525; Riechmann et al. (1988) Nature 332:323-329; and Presta (1992) Curr. Op. Struct. Biol. 2:593-596. The use of humanized antibodies obviates potential problems associated with the immunogenicity of murine constant regions.

Before being used according to the invention, antibodies according to the invention may be purified. Methods for antibody purification are well known in the field of biomedical research, some of which rely on the unique characteristics of the antibodies to be purified, whereas others are standard protein separation techniques suitable for a broad range of applications.

Salt fractionation can be used as an initial step to separate desired antibodies from other unwanted proteins. The preferred salt is ammonium sulfate, which precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The desired antibody is precipitated at an appropriate ammonium sulfate concentration according to its hydrophobicity and is then solubilized in a buffer with the excess salt removed if necessary, through either dialysis or diafiltration. Other methods that rely on solubility of proteins, such as cold ethanol precipitation, are well known to those of skill in the art and may also be used to prepare an antibody fraction from a protein mixture, such as a serum.

Based on a predicted molecular weight, an antibody can be isolated from proteins of greater and lesser sizes using ultrafiltration through membranes of different pore sizes (for example, Amicon or Millipore membranes). As a first step, a protein mixture (e.g., a serum or a cell culture supernatant) is ultrafiltered through a membrane with a pore size that has a lower molecular weight cut-off than the predicted molecular weight of the desired antibody. The retentate of the ultrafiltration is then ultrafiltered against a membrane with a molecular cut-off greater than the predicted molecular weight of the desired antibody. The antibody will pass through the membrane into the filtrate, which can then be processed in a next step of column chromatography.

Antibodies according to the invention can also be separated from other proteins including other antibodies on the basis of their size, net surface charge, hydrophobicity, and affinity for ligands. Column chromatography is a frequently used method. For example, antibodies can be isolated from other non-antibody proteins using a column with immobilized protein A or protein G, which are bacterial cell wall proteins that bind to a domain in the Fc region of antibodies. Furthermore, antibodies against different antigens can be separated based on their distinct affinity to these antigens, which are immobilized to a column in a preferred format of column chromatography for antibody purification. All of these methods are well known in the art, and it will be apparent to one of skill that chromatographic techniques can be performed at any scale and using equipment from many different manufacturers (e.g., Pharmacia Biotech).

The present invention also relates to the use of an antibody as defined above for detecting a CRMP2 which is phosphorylated on tyrosine 479, an optionally further on serine 465. Said detection may be carried out using an suitable immunodetection technique enabling visualizing the binding of an antibody. Such detection techniques are well-known from the one skilled in the art and include immunohistocytochemistry, immunofluorescence, immunoprecipitation, western-immunobloting, chimioluminescence, colorimetric and radiolabelling techniques.

In a preferred embodiment, the use of the antibody according to the invention is for detecting a CRMP2 which is further in a cleaved form.

### Methods for In Vitro Prognosis, Diagnosis and/or Monitoring

The present invention relates to a method for in vitro prognosis, diagnosis and/or monitoring of an inflammatory disease of the central nervous system in a subject, said method comprising detecting, in a sample of cells of the immune system from the subject, the presence of Y479-phosphorylated CRMP2, wherein the detection of the presence of Y479-phosphorylated CRMP2 is indicative of an inflammatory disease of the central nervous system.

The present invention also describes an in vitro method for detecting an inflammatory disease of the central nervous system in a subject, said method comprising detecting in vitro in a sample of cells of the immune system taken from the subject, the presence of Y479-phosphorylated CRMP2, wherein the detection of the presence of Y479-phosphorylated CRMP2 is indicative of an inflammatory disease of the central nervous system.

As used herein, a “diagnostic method” or “diagnosis” refers to a method for determining whether a subject suffers from a pathology.

As used herein, a “prognostic method” or “prognosis” refers to a method for determining whether a subject is likely to develop a pathology.

As used herein, “monitoring method” refers to a method for determining the evolution of a pathology in a subject.

As used herein, “cells of the immune system” encompass cells of the innate and adaptative immune response, in particular T and B lymphocytes, dendritic cells, monocytes and natural killer cells.

As used herein, a “sample” refers to a part of a bigger set. Preferably, the sample of cells of the immune system according to the invention is taken from the blood or the brain of a subject, and/or include in particular subpopulations of blood cells and the like. Preferably, the sample of cells of the immune system according to the invention comprises or consists in T lymphocytes.

In a particular embodiment, in the above defined method, said Y479-phosphorylated CRMP2 is in a full-length form. In another particular embodiment, in the above defined method, said Y479-phosphorylated CRMP2 is in a cleaved form. In these embodiments, said Y479-phosphorylated CRMP2 may be further phosphorylated on serine 465. Preferably, when Y479-phosphorylated CRMP2 is in a cleaved form, it is further phosphorylated on serine 465. Additionally, CRMP2 may be further phosphorylated on other phosphorylation sites such as serine 522, threonine 509, threonine 514 and/or threonine 555.

In the context of the invention, the detection of the presence of Y479-phosphorylated CRMP2 in a sample of cells of the immune system of a subject may be carried out by any suitable technique enabling visualizing the presence of a protein. In particular, it can be performed using a compound displaying a specific affinity for CRMP2, more preferably for Y479-phosphorylated CRMP2 or for S465/Y479-phosphorylated CRMP2. Such suitable compounds include in particular antibodies and aptamers. Preferably, Y479-phosphorylated CRMP2 is detected with an antibody, preferably, with an antibody specific of Y479-phosphorylated CRMP2 as defined above. Accordingly, suitable techniques enabling visualizing the presence of said protein encompass any technique of immunodetection enabling visualizing the binding of an antibody. Such detection techniques are well-known from the one skilled in the art and include immunohistocytochemistry, immunofluorescence, immunoprecipitation, western-immunobloting, chimioluminescence, colorimetric and radiolabelling techniques.

As used herein, a “subject” refers a human or non-human mammal (such as a rodent (mouse, rat), a feline, a canine, or a primate). Preferably, the subject is a human.

The present invention also relates to the antibody as defined above for use in the prognosis, diagnosis and/or monitoring of an inflammatory disease of the central nervous system.

### Methods of Treatment

The present invention also relates to the antibody as defined above for use for decreasing immune cells migration. In particular the invention relates to an antibody specific of a CRMP2 which is phosphorylated on tyrosine 479 for use for decreasing immune cells migration.

As used herein, “immune cells” encompass cells of the innate and adaptative immune response, in particular T and B lymphocytes, dendritic cells, monocytes and natural killer cells. Preferably, immune cells according to the invention are T lymphocytes.

In the context of the invention, “immune cells migration” refers to the trafficking of immune cells from lymphoid organs to effector sites. As used herein, “decreasing immune cells migration” means slowing the rate of migration of immune cells, decreasing the number of immune cells which migrate from lymphoid organs to the effector site, or inhibiting the migration of immune cells to the effector site. Preferably, in the context of the invention, the effector site is the brain.

The present invention also relates to the antibody as defined above for use in the treatment of an inflammatory disease of the central nervous system as defined above.

The present invention also relates to a method of treatment of an inflammatory disease of the central nervous system comprising the administration of a therapeutically effective amount of the antibody as defined above in a subject in need thereof.

The term “treating” or “treatment”, as used herein, means reversing, alleviating, inhibiting the progress of, or preventing the disorder or condition to which such term applies, or one or more symptoms of such disorder or condition.

According to the invention, the term “subject” or “subject in need thereof” is intended for a human or non-human mammal (such as a rodent (mouse, rat), a feline, a canine, or a primate).

The term “therapeutically effective amount” is meant for a sufficient amount of antibody in order to treat said disease, at a reasonable benefit/risk ratio applicable to any medical treatment. It will be understood, however, that the total daily usage of the antibodies of the present invention will be decided by the attending physician within the scope of sound medical judgment. The specific therapeutically effective dose level for any particular patient will depend upon a variety of factors including the disorder being treated and the severity of the disorder, activity of the specific antibody employed, the specific composition employed, the age, body weight, general health, sex and diet of the patient, the time of administration, route of administration, and rate of excretion of the specific antibody employed, the duration of the treatment, drugs used in combination or coincidental with the specific antibody employed, and like factors well known in the medical arts. For example, it is well known within the skill of the art to start doses of the compound at levels lower than those required to achieve the desired therapeutic effect and to gradually increase the dosage until the desired effect is achieved.

The antibody of the invention may be used in combination with any other therapeutical strategy for treating an inflammatory disease of the central nervous system.

### Antagonists of the CXCR4 Receptor and Uses Thereof

The present inventors have demonstrated that the phosphorylation of CRMP2 on tyrosine 479 was controlled by the CXCR4 receptor. Accordingly, inhibiting the CXCR4 receptor would prevent the phosphorylation of CRMP2 on tyrosine 479, and accordingly decreasing immune cells migration.

The present invention thus also relates to an antagonist of the CXCR4 receptor for use for decreasing immune cells migration as defined above. Preferably, the antagonist of the CXCR4 receptor is for use for decreasing T lymphocytes migration.

As used herein, the “CXCR4 receptor” or “fusin” refers to a CXC chemokine receptor which is an α-chemokine receptor specific for stromal-derived-factor-1 (SDF-1 also called CXCL12).

In the context of the invention, an “antagonist of the CXCR4 receptor” refers to a compound which inhibits, directly by binding to the CXCR4 receptor, or indirectly, the signalization cascade downstream the CXCR4 receptor. Examples of antagonists of the CXCR4 receptor include antibodies specific of the CXCR4 receptor, AMD070, AMD3100 (or Plerixafor), AMD3465, 4F-benzoyl-TN14003 (or T140), KRH-3955, and bicyclams.

The present invention also relates to the antagonist as defined above for use in the treatment of an inflammatory disease of the central nervous system as defined above.

A method of treatment of an inflammatory disease of the central nervous system comprising the administration of a therapeutically effective amount of the antagonist of the CXCR4 receptor as defined above in a subject in need thereof is also an object of the present invention.

The antagonist of the invention may be used in combination with any other therapeutical strategy for treating an inflammatory disease of the central nervous system. In particular, the antagonist of the invention may be used in combination with an antibody of the invention.

The invention is further illustrated by the following figures and examples.

### BRIEF DESCRIPTION OF THE FIGURES

FIG. 1 shows histograms representing the number of Jurkat cells (in %), adhering to collagen I-coated slides, with polarized CRMP2 0, 2, 5 or 10 min after treatment with CXCL12 (100 ng/mL) and fixation. CRMP2 was observed with anti-CRMP2-Cter antibody.

FIG. 2 shows histograms representing the number of Jurkat cells (in %), adhering to collagen I-coated slides, with polarized CRMP2 after treatment with CXCL12 (100 ng/mL) alone or after treatment with CXCL12 and with the CXCR4 antiagonist AMD3100.

FIG. 3 shows the result of Western Blots performed on whole cell lysates, cytosol or cytoskeleton fractions of Jurkat cells treated with (+) or without (−) CXCL12 (100 ng/ml) for 10 or 30 min, lyzed, and subjected to sub-cellular fractionationalion. The Western Blots were performed using anti-CRMP2-pep4 or anti-CRMP-C-ter antibodies. The open arrow shows the full-length form of CRMP2 and the black arrow shows the cleaved form of CRMP2. Western Blots were also performed with anti-vimentin, anti-Erk1/2 and anti-pErk1/2 antibodies. The numbers on the left of the Western Blots represent the molecular weight (in kDa). The numbers bellow the Western Blots represent the relative value of the signal intensity obtained with the treated cells compared to the signal intensity obtained with the untreated cells.

FIG. 4 shows the result of Western Blots performed on whole cell lysates, un-phosphorylated proteins (flow-through) or phosphorylated proteins (eluate) of Jurkat cells treated with CXCL12 (100 ng/ml) for 0, 2, 5, 10 or 30 min, lyzed, and subjected to phosphorylated form enrichment. The Western Blots were performed using anti-CRMP2-pep4 or anti-CRMP-C-ter antibodies. The open arrow shows the full-length form of CRMP2 and the black arrow shows the cleaved form of CRMP2. Western Blots were also performed with anti-Erk1/2 and anti-pErk1/2 antibodies. The numbers on the left of the Western Blots represent the molecular weight (in kDa).

FIG. 5 shows the result of Western Blots performed on cell lysates of Jurkat T-cells treated with CXCL12 (100 ng/ml) for 0, 1, 2, 4, 6, 10, 15 or 30 min. The Western Blots were performed using anti-CRMP2-pSer522 antibodies, anti-CRMP2-pThr509/514 antibodies, anti-CRMP-C-ter antibodies, anti-pErk1/2 antibodies and anti-Erk1/2 antibodies. The numbers on the left of the Western Blots represent the molecular weight (in kDa). The numbers bellow the Western Blots represent the relative value of the signal intensity obtained with the treated cells compared to the signal intensity obtained with the untreated cells.

FIG. 6 shows the result of Western Blots performed on cell lysates of Jurkat T-cells treated with CXCL12 (100 ng/ml) for 0, 1, 2, 4, 6, 10, 15 or 30 min. The Western Blots were performed using anti-Cdk5-pTyr12 antibodies, anti-Cdk5-pSer159 antibodies, anti-Cdk5 antibodies, anti-GSK-3α-pTyr279 antibodies, anti-GSK-3β-pTyr216 antibodies, anti-GSK-3α antibodies and anti-GSK-3β antibodies. The numbers on the left of the Western Blots represent the molecular weight (in kDa). The numbers bellow the Western Blots represent the relative value of the signal intensity obtained with the treated cells compared to the signal intensity obtained with the untreated cells.

FIG. 7 shows the ribbon diagram of the structure of cleaved CRMP2 from residues 15 to 489. The inset shows the surface representation of cleaved CRMP2, indicating the surface exposure of residues R467, P470 and P473 (dark grey).

FIG. 8 shows the result of Western Blots performed on cell lysates of primary T-lymphocytes or Dev neural cells incubated with sepharose-4B beads coupled to CRMP2-GST or GST. The Western Blots were performed using anti-Yes antibodies, anti-CRMP2 antibodies and anti-GST antibodies. The numbers on the left of the Western Blots represent the molecular weight (in kDa).

FIG. 9 shows the results of in vitro kinase assays performed with (+) or without active recombinant Yes and with (+) or without recombinant CRMP2 in the presence (+) or absence of ATP. Western Blots were performed using anti-phospho tyrosine residues antibodies and anti-CRMP2 antibodies. The numbers on the right of the Western Blots represent the molecular weight (in kDa).

FIG. 10 shows the result of Western Blots performed on cell lysates of Jurkat cells treated with CXCL12 (100 ng/ml) for 0, 1, 2, 4, 6, 10, 15 or 30 min. The Western Blots were performed using anti-CRMP2-pTyr479 antibodies, anti-CRMP-C-ter antibodies, anti-CRMP2-pep4 antibodies, anti-Src-pTyr416 antibodies and anti-Src antibodies. The open arrow shows the full-length form of CRMP2 and the black arrow shows the cleaved form of CRMP2. The numbers on the left of the Western Blots represent the molecular weight (in kDa). The numbers bellow the Western Blots represent the relative value of the signal intensity obtained with the treated cells compared to the signal intensity obtained with the untreated cells.

FIG. 11 shows histograms representing the number of Jurkat cells (in % compared to the number of transfected cells), transfected with CRMP2Flag-WT (hashed bars) or with CRMP2Flag-Y479F mutants (white bars), adhering to collagen I-coated slides, with polarized CRMP2 after treatment with CXCL12 (100 ng/mL) and fixation. CRMP2 was observed with anti-Flag antibodies.

FIG. 12 shows histograms representing the number of Jurkat cells, transfected with an empty vector (dot bar), CRMP2Flag-WT (hashed bar) or CRMP2Flag-Y479F (white bar) plasmids, that transmigrate towards CXCL12 in Transwell chambers.

FIG. 13 shows histograms representing the number of Jurkat cells, transfected with CRMP2Flag-WT (hashed bar) or CRMP2Flag-Y479F (white bar) plasmids, that migrate on neural tissue when spotted close to hippocampal organotypic slices.

### EXAMPLE 1

The following example demonstrates that the phosphorylation of CRMP2 on tyrosine 479 residues in crucial in T cells migration.

Material and methods

### Cells and Antibodies

The Jurkat T cell line was cultured in RPMI 1640 complemented with 10% fetal calf serum. Primary T lymphocytes selected from the blood of a healthy donor were cultured for one to two weeks in RPMI complemented with 10% AB human serum and IL2 (20 U/mL).

Rabbit polyclonal antibodies recognizing both full-length and cleaved CRMP2 forms have been described in Rogemond et al. (2008) J. Biol. Chem. 283:14751-14761. The peptide sequences used to generate C-ter and pep4 antisera were localized between AA557-572 and AA454-465 in the CRMP2 sequence, respectively. Antibodies were purified by affinity chromatography on the corresponding immobilized peptide. Sheep antisera recognizing CRMP2-pSer522 and CRMP2-pTyr509/514 were from Kinasource Limited (Dundee, UK). The rabbit polyclonal antibody produced by the inventors was raised against the peptide AA470-483 phosphorylated on Y479 (CRMP2-pY479) and purified in a two steps method by affinity chromatography on the corresponding immobilized peptide (step1: elimination of antibody anti un-phosphorylated peptide, step 2: purification of anti phosphorylated peptide). ELISA performed against CRMP2-Y479 and CRMP2-pY479 peptides showed the specificity of the anti-CRMP2-pY479 antibody produced by the inventors. In addition, treatment of T cell lysate with phosphatase CIP significantly reduced the positive signal in Western blotting. Rabbit polyclonal antibody anti Yes kinase was from Upstate. Mouse anti Vimentin/LN6 was from Calbiochem. Anti-Erk and phospho-Erk antibodies from Cell Signaling recognized un-phospho- and phospho-p44/42 MAP Kinase (Erk1 and Erk2). Anti-Cdk5, CDK5-pTyr15, Cdk5-pSer159 and Src antibodies were from Santa Cruz Biotechnology. The rabbit anti phospho-Src family was from Cell Signaling and recognized phosphorylated Tyr416 on Src, Lyn, Fyn, LCK, Hck and Yes. Rabbit anti-GSK-3 was from Chemicon International. Mouse anti-pGSK-3 from Upstate Millipore recognized the active forms of GSK-3α (pTyr279) and GSK-3β (pTyr216). Magnetic phospho enrichment beads (TALON® PMAC) were purchased from Clontech.

### Plasmids and Constructs

The CRMP2-Flag-wt plasmid has been described in Rogemond et al. (2008) J. Biol. Chem. 283:14751-14761. Briefly, full-length CRMP2 was amplified by PCR and inserted directionally into the pCMV2-FLAG vector (Sigma, l'Isle d'Abeau, France).

Mutation: A two steps PCR procedure was used to generate the CRMP2-Y479F mutant. First, a C-terminal fragment (471-572) containing the Y479-F mutation was generated using a reverse primer introducing an EcoRI site at the 3′ end and a forward primer with the codon: Y479 (TAC) substituted with F (TTC). Next, this mutated fragment was used as a reverse primer in the second PCR reaction with a wild type forward primer introducing a HindIII site at the 5′ end. The final PCR product was cloned into the HindIII and EcoRI sites of the pCMV2-Flag vector and the DNA sequence of the mutant was verified by sequencing.

Transfection: Jurkat T cells were transfected with CRMP2-Flag-wt, CRMP2-Flag-Y479F and empty-Flag plasmids using Amaxa Nucleofector technology (Köln, Germany), according to the manufacturer's instructions. T cells were used 18 h after transfection. Transfected cells were visualized by immunostaining with anti-Flag antibody. The percentage of transfection reached 40-50% for most of the Flag constructs.

### Immunocytochemistry

The CRMP2 forms, Yes kinase and intermediary filament vimentin were detected by indirect immunofluorescence on Jurkat and primary T-cells adhered to collagen I-coated slides (20 μg/ml) and fixed following treatment (acetone −20° C.; 10 min). Cells were incubated with specific antibody (1 h, 37° C.) then with Alexa 488- or 546-conjugated anti-mouse or anti-rabbit or anti-sheep IgG antibodies (1 h, 37° C.) and examined using the Axioplan II fluorescence microscope (Carl Zeiss). Nuclear counter-staining was performed using a fluorescent DNA intercalant, 4′, 6′-diamidino-2-phenylindole (DAPI, Boehringer Mannheim).

### Protein Interaction Assay (Pull-Down GST-CRMP2)

One hundred microliters of Jurkat cell lysate, prepared as above, were added either to 80 μl of GST-CRMP2 protein fusion or to 80 μl of GST protein coupled with glutathione-Sepharose 4B (Pharmacia Biotech) (1 h at 4° C.). GST-CRMP2 and GST beads were washed four times (50 mM Tris (pH 7.4), 1 mM EDTA, 150 mM NaCl, and 0.5% Nonidet P40), and proteins bound to CRMP-2 or to GST beads alone were eluted. GST (in GST beads), Yes and CRMP2 (in GST-CRMP2 beads) and GST (in GST and GST-CRMP2 beads) were revealed by Western blotting.

### Western Blotting

Following CXCL12 treatment, cells were lysed in homogenization buffer (Tris 20 mM, EDTA 1 mM, EGTA 5 mM, sucrose 10%, pH 7.4) complemented with phosphatase inhibitors (Na fluoride 5 mM, Na pyrophosphate 1 mM, b-glycerophosphate 1 mM, orthovanadate 1 mM) and with protease inhibitor cocktail Complete™ (Roche). Lysates were submitted to ultrasound to dissociate cell aggregates and total proteins measured by Lowry assay (Bio-Rad). Protein samples (10-20 μg) were subjected to sodium dodecyl sulfate-polyacrylamide gel electrophoresis (SDS-PAGE) under reducing conditions and transferred to nitrocellulose membranes (BA85; Schleicher & Schuell Microscience) previously incubated with blocking solution (PBS, 0.1% Tween 20, 5% nonfat dried milk, 1 h) and blotted against specific antibody (overnight, 4° C.), followed by incubation (1 h, room temperature) with rabbit and sheep IgGs antibody coupled with horseradish peroxidase (HRP) and a chemiluminescence (ECL) detection system (Covalab Lyon, France). Densitometric quantification of the immunoblot band was performed using Image Quant (Molecular dynamics) and the data expressed as ratios to the amount detected before any treatment.

### Database and Structure Analysis

The prediction programme PROSITE (http://us.expasy.org/prosite) was used to identify the putative tyrosine kinase site on CRMP2. The structure of CRMP2 was modeled, based on the coordinates available for CRMP2 chain D (Stenmark et al. (2007) J. Neurochem. 101:906-917) (protein Data Bank entry 2GSE), using Viewerlite/4.2 (Accelrys).

### Cloning and Expression of CRMP2

The coding sequence for human CRMP-2 (NM_{—}1386) was subcloned into the expression vector pEt21b (Novagen), resulting in a construct with an N-terminal hexahistidine tag. The plasmid was transformed into Escherichia coli BL21(DE3) cells. For expression, cells were grown in 1500 mL Terrific Broth (containing 7% glycerol, 50 μg/mL kanamycin and 100 μL BREOX) in bubble flasks. Cells were grown at +37° C. until an optical density of 2.5 at 6 00 nm was reached. The cultures were cooled to +18° C. for 1 h in a water bath. The expression of CRMP-2 was induced by the addition of 0.5 mmol/L IPTG, and expression was allowed to continue overnight at +18° C. Cells were harvested by centrifugation, and the pellets were suspended in lysis buffer (20 mM Tris, 500 mM NaCl, 1 mM DTT, 20% glycerol, 0.1% triton, 10 mM imidazole) supplemented with Complete EDTA-free protease inhibitors (Roche, Basel, Switzerland) and 2000 U of benzonase. The solution was sonicated for several cycles on ice. The samples were centrifuged at 14,000 g for 30 min at 4° C., and the supernatants were incubated with 1.5 ml Ni-NTA resin 50% re-suspended in lysis buffer (QIAGEN) at 4° C. for 90 minutes. His-tagged proteins were purified from Ni resin in a wash buffer (20 mM Tris, 500 mM NaCl, 1 mM DTT, 20% glycerol, 0.1% triton, 20 mM imidazole) and were eluted with elution buffer (wash buffer+150 mM imidazole) in 1 ml fractions. Fractions were evaluated by SDS-PAGE.

### Yes In Vitro Kinase Assay

Prior to the assay, His-tagged CRMP2 was dialyzed in buffer (40 mM MOPS, 0.5 mM EDTA, 5% glycerol) overnight at 4° C. using the Float-A-lyzer technology (Interchim), according to manufacturer instructions. For the Yes kinase assay, 0.6 μg of dialyzed His-tagged CRMP2 were incubated with 20 ng of recombinant full-length human Yes (Millipore) diluted beforehand in enzyme dilution buffer (20 mM MOPS pH 7, 1 mM EDTA, 0.01% Brij, 0.1% β-mercaptoethanol, 5% glycerol). The reaction was allowed in 50 μl of reaction buffer (8 mM MOPS, 0.2 mM EDTA, 30 mM MgCl₂, 2 mM EGTA, 10 mM β-glycerophosphate, 0.4 mM Na₃VO₄, 0.4 mM DTT, 200 μM ATP) at 30° C. for 30 minutes. The reaction was stopped with loading buffer and the mixture was resolved on SDS-PAGE gels.

### Transmigration Assay

T cell transmigration was performed with Jurkat T cells both in micro-Transwell systems (Costar Transwell Supports-A) and in organotypic cultures of mouse brain (B).

A: Transmigration was performed in triplicate in Transwell systems (Boyden chamber, Costar, 5-μm diameter pore size membrane), as described Vincent et al. (2005) J. Immunol. 175:7650-7660. Briefly, the T-cell preparation (3×10⁵ cells/well) was added in the upper chambers and CXCL12 in the lower compartment (10 ng/mL). Following a 2 h incubation at 37° C., cells mi grating in the lower chambers were counted under the microscope (at least 30 fields examined).

B: T-cell transmigration on neural tissue was assayed on hippocampal cultures prepared as follows. Hippocampi from postnatal (P7) C57BL6 mice were dissected and placed immediately in cold Gey's balanced solution supplemented with glucose (6.5 mg/ml). Four hundred micrometer slices were cut perpendicularly to the septotemporal axis of the hippocampus using a McIllwain tissue chopper. Slices were carefully trimmed for excess tissue, and 6 slices were placed immediately on 30 mm semi-permeable membrane inserts (Millicell-CM, Millipore) in a 6-well plate, each well containing 1 ml of culture medium. The culture medium consisted of 50% Minimum Essential Medium (Gibco), 25% Hank's balanced salt solution, 25% heat-inactived horse serum (Gibco), 1% I-glutamine 200 mM (Gibco) and 6.5 mg/ml D-glucose. Plates were incubated at 37° C. and 5% CO₂. The culture medium was exchanged twice a week. Jurkat T cells (1×10⁶ cells per slice) stained ex vivo using the vital fluorochrome carboxyfluoroscein succinimidyl ester/CFSE (1 mM, 5 min, 37° C.) were spotted close to the hippo campus slices (one week culture). Following 18 h contact at 37° C., slices were extensively washed with D-MEM, fixed with ethanol (10 min, 4° C.) and incubated with DAPI for nuclear counter-staining. The number of infiltrating lymphocytes, counted under fluorescence microscopy, decreased in T-cell populations transfected with CRMP2-Y479-F.

### Statistical Analysis

Statistical significance in comparing two means was tested with the unpaired Student's t test, p values<0.05 were considered significant. In the migration test, the number of migratory lymphocytes was counted by light microscopy (15-20 microscope fields per condition—2 or 3 independent experiments) and data expressed as the mean number of migratory lymphocytes per field.

### Results

### CXCL12 Induces CRMP2 Polarization at the T Lymphocyte Uropod.

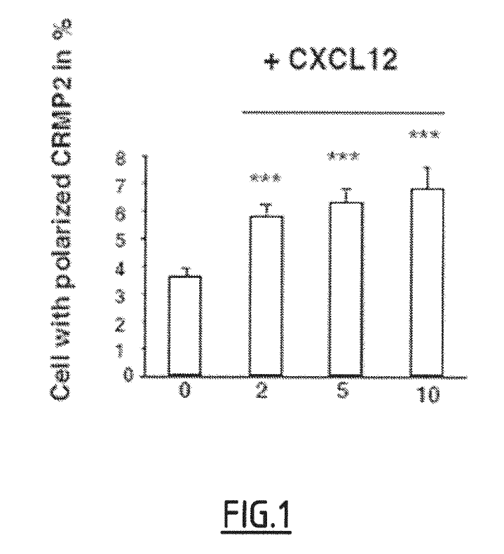

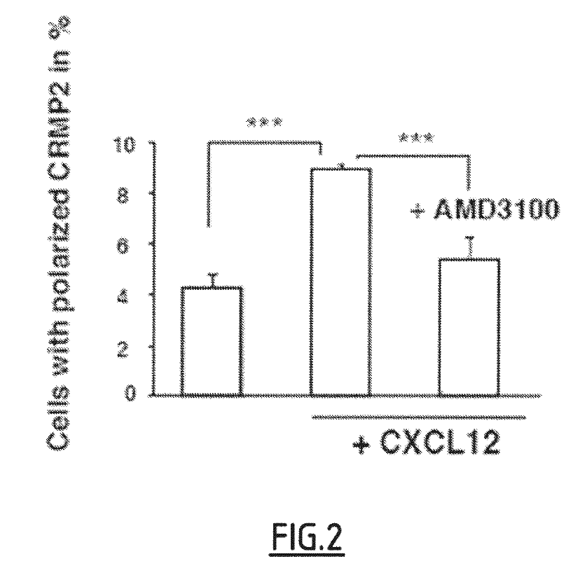

To define a link between chemokines and CRMP2, the inventors first examined the localization of CRMP2 in Jurkat T-cells under CXCL12 signaling. They used two different anti-CRMP2 antibodies (anti-C-ter and anti-pep4) that recognize the full-length and cleaved products of CRMP2. An immunofluorescence study of untreated T-cells revealed that CRMP2 was found within the T-cell cytoplasm as punctate dots. Under CXCL12 treatment, CRMP2 moved to the cell trailing edge within 2 minutes and showed quasi-exclusive uropod localization in most polarized cells after 10 minutes treatment. This phenomenon of CRMP2 polarization was still observed after 30 minutes of treatment. Un-treated Jurkat T-cells showed an asymmetrical CRMP2 distribution in cells, but increases of 1.6 to 2 fold were observed after CXCL12 treatment (FIG. 1). Similar CRMP2 re-localization was observed with anti-pep4 antibody staining. In addition CRMP2 distribution to the uropod was concomitant with the re-localization of vimentin, which was quickly redistributed at the trailing edge of polarizing T-cells. Interestingly, CRMP2 re-localization was reversed to a great extent (35% decrease) in the presence of AMD3100, an antagonist of the CXCL12 receptor (CXCR4), consequently confirming the specificity of the CXCL12-induced response (FIG. 2). These results supported the idea that chemokines can induce a dynamic re-localization of CRMP2 in T lymphocytes in concert with vimentin, namely in the flexible uropod structure.

### CXCL12 Modulates CRMP2 Binding to the Cytoskeleton.

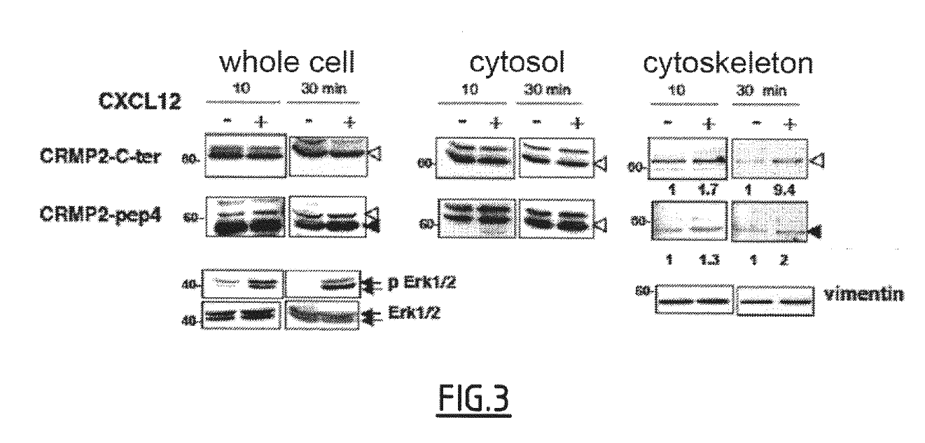

It is well known that T-cell uropods are rich in vimentin and microtubules (Serrador et al. (1999) Trends Cell Biol. 9:228-233), two cytoskeletal elements that have both been described as CRMP2 binding partners (Vincent et al. (2005) J. Immunol. 175:7650-7660; Gu et al. (2000) J. Biol. Chem. 275:17917-17920) and actors in T-lymphocyte polarization and migration (Krummel et al. (2006) Nat. Immunol. 7:1143-1149). This led the inventors to hypothesize that CXCL12 could modulate CRMP2 binding to the cytoskeleton to promote T-cell motility. Following CXCL12 treatment (100 ng/mL, 10 and 30 min), sub-cellular fractionation was performed on Jurkat T-cell extracts to isolate cytoskeletal elements and associated proteins from the cytosol fraction. Identification of the sub-cellular fractions using antibodies against vimentin, tubulin PARP and Hsp90 indicated that there was no contamination between cytoskeletal and cytosolic fractions. The cytoskeletal fraction displayed the intermediate filement vimentin but was free from tubulin, probably due to de-polymerization as it is found in the cytosol. Different fractions were then subjected to Western blotting using anti-CRMP2 antibodies. In whole cell lysates of untreated cells, anti-C-ter antibody revealed CRMP2 bands corresponding to the previously described full-length CRMP2 (62 kDa) and bands with higher molecular weight (FIG. 3). Anti-pep4 antibody mainly recognized a 58 kDa band, corresponding to the cleaved form of CRMP2, as reported in neural cells (Rogemond et al. (2008) J. Biol. Chem. 283:14751-14761). The affinity of anti-pep4 antibody was higher for the cleaved form than for the full-length form. After CXCL12 treatment, the efficiency of which was assessed by Erk1/2 phosphorylation (FIG. 3), no difference in CRMP2 expression was detectable in whole cell lysates. However, the distribution of CRMP2 forms differed according to the T-cell compartment examined. Full-length CRMP2 and higher molecular weight bands were found in the cytosolic fractions. These did not show major alterations under CXCL12 treatment. CRMP2 was also found, to a lesser extent, in the cytoskeletal fractions as 62 kDa full-length and 58 kDa cleaved forms. Interestingly, the expression of both forms was enhanced following CXCL12 treatment. It should be noted that the majority of cleaved CRMP2 was found in the nucleus, and was not modified under CXCR4 activation. These results showed that CRMP2 was distributed in the cytoskeletal compartment of T lymphocytes and that CXCL12 had the ability to alter this distribution, enhancing CRMP2 association with cytoskeletal elements.

### CXCL2 Increases CRMP2 Phosphorylation.

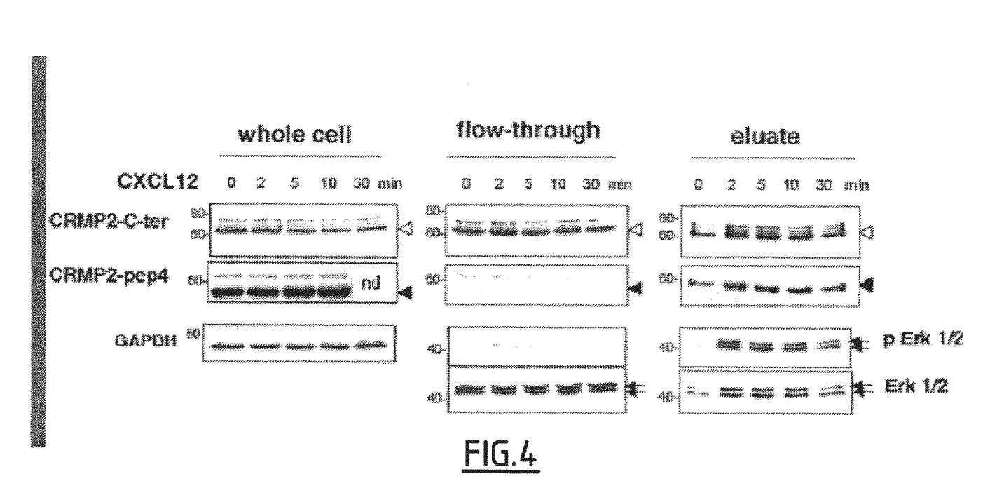

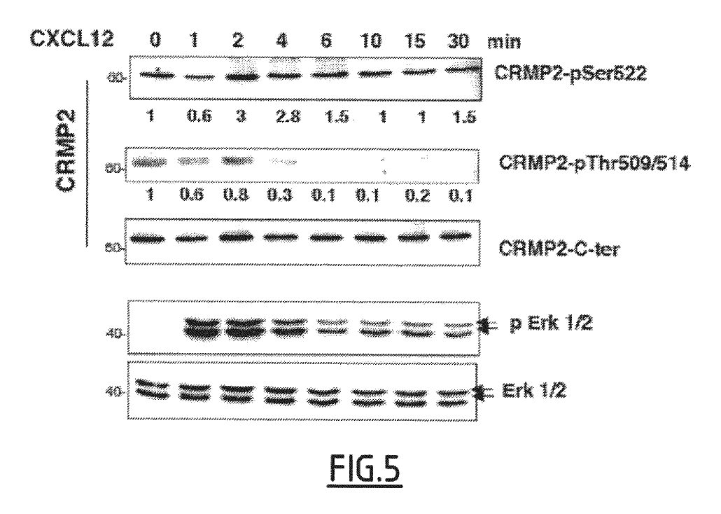

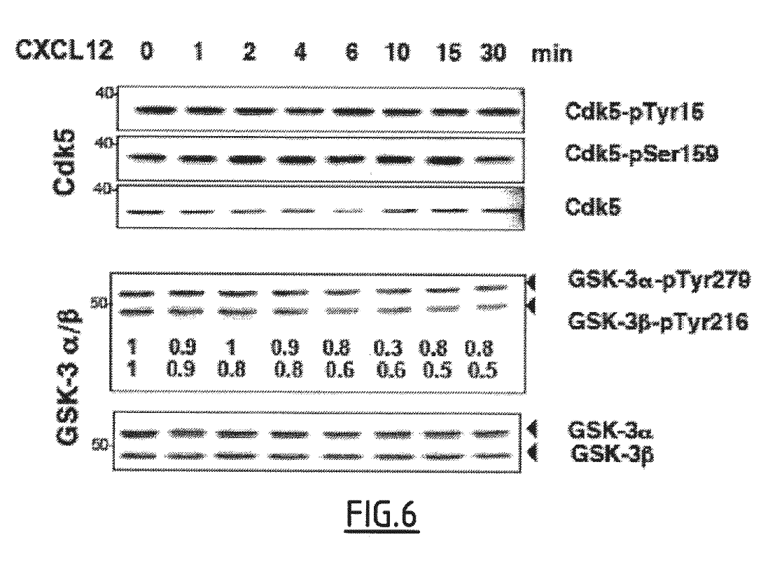

Functional regulation of CRMP2 in neural cells is mainly dependent on its phosphorylation state, notably via GSK-3β and Cdk5 kinase activity (Uchida et al. (2005) Genes Cells 10:165-179). The inventors therefore studied whether, in T lymphocytes, CXCL12 could modify CRMP2 binding to the cytoskeleton through modulation of its phosphorylation. To evaluate CRMP2 phosphorylation, the inventors performed a phosphoprotein enrichment assay (TALON® PMAC, Clonetech) on whole cell extracts of Jurkat T-cells following CXCL12 treatment (100 ng/ml) and carried out immunoblotting on the non-phosphorylated (flow through) and phosphorylated (eluate) fractions by Western blotting at 2, 5, 10 and 30 min post-treatment. The full-length CRMP2 forms revealed by the anti-C-ter antibody were present in both the un-phosphorylated and phosphorylated fractions (FIG. 4). In contrast, the cleaved form of CRMP2 was only found in the phosphorylated protein pool, indicating that this form is mostly phosphorylated. CXCL12 treatment rapidly increased the level of CRMP2 phosphorylated forms, peaking at 2 min post treatment and still high at 30 min. The efficiency of the phosphoprotein enrichment procedure was ascertained by phospho-Erk1/2 immunoblotting, which confirmed the specific presence of phosphorylated proteins in the eluate and at the same time, the increase following CXCL12 treatment. Similar experiments performed on primary T-lymphocytes isolated from healthy donors showed similar observations. A more precise evaluation of CRMP2 phosphorylation in response to CXCL12 was carried out using anti-CRMP2-pSer522 and anti-CRMP2-pThr509/514 antibodies recognizing two sites targeted by Cdk5 and GSK-3 kinases, respectively (FIG. 5). Immunoblotting of Jurkat cell lysates showed that CRMP2-pSer522 and CRMP2-pThr509/514 were present as full length 62 kDa CRMP2 in T-cells and were variously expressed during chemokine treatment, the efficiency of which was ascertained by phospho-Erk1/2 detection. While Ser522 phosphorylation was found at relatively low levels, Thr509/514 phosphorylation decreased quickly by 4 min and was undetectable thereafter. This was consistent with the activity of Cdk5 and GSK-3 kinases evaluated by the detection of Cdk5-pTyr15, Cdk5-pSer159, GSK-3α-pTyr279 and GSK-3β-pTyr216, the active forms of these kinases (FIG. 6). Cdk5 displayed a stable level of phosphorylation on Tyr15 and Ser159, reflecting a conserved level of Cdk5 activation. In contrast, GSK-3 exhibited dephosphorylation mainly detected on the GSK-3β isoform, revealing a decreased activity starting at 4 min post-treatment. Taken together, these results first revealed, as previously described in neural cells, that the CRMP2 residues Ser522 and Thr509/514 could be phosphorylated in T lymphocytes. More importantly, they demonstrate that CXCL12 triggers a signaling cascade leading to differential modulation of CRMP2 phosphorylation of these residues, namely with a net decreased phosphorylation on Thr509/514. Intriguingly, these modulations were mainly detected on the full-length CRMP2 forms, while phosphoprotein enrichment assays (FIG. 4) showed a strong phosphorylation of the cleaved CRMP2 form following chemokine treatment. This led us to suspect the participation of an additional phosphorylated target in the response of CRMP2 to CXCL12.

### Tyrosine 479 is a New Phosphorylation Residue in CRMP2 Sequence

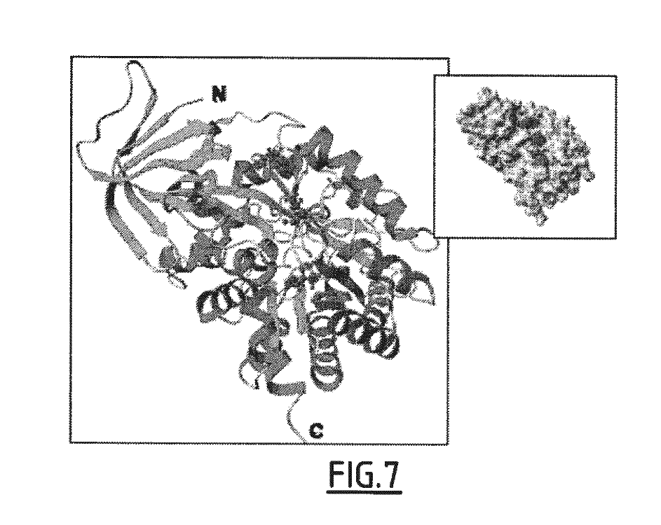

It is known that CXCL12 triggers a tyrosine phosphorylation cascade in T-lymphocytes, which involves the serial recruitment and activation of tyrosine kinases including Lck, ZAP-70 and Itk (Patrussi et al. (2008) Immunol. Lett. 115:75-82). The inventors therefore searched for tyrosine target residues potentially modulated under chemokine treatment by analyzing CRMP2 protein sequences. A database study of the 572 amino acids identified tyrosine 479 (Y479) as a potential new phosphorylation residue, located in the phosphotyrosine consensus motif KxxxDxxY within residues 472-479 (FIG. 7). In addition, inspection of this region also showed the presence, close to Y479, of a putative SH3-binding motif of the form RxxPxxP within residues 467-473. In order to assess the accessibility of these sequences to binding protein partners, the inventors evaluated the position of both Y479 and the SH3-binding motif within the known structure of CRMP2 (FIG. 7) based on the coordinates available for fragment 15-489 (Stenmark et al. (2007) J. Neurochem. 101:906-917). Surface exposure representation of this CRMP2 form revealed that, in contrast to Y479, the putative SH3-binding motif was exposed, suggesting a possible binding with SH3-domain bearing proteins (FIG. 7 insert). It has been shown that interaction with the SH3 domain-binding motif induces protein conformational changes (Martinez and Serrano (1999) Nat. Struct. Biol. 6:1010-1016), so the latter could be the basis of subsequent Y479 exposure. These observations suggested Y479 as a major putative phosphorylation tyrosine within the CRMP2 sequence.

### CRMP2 Tyrosine-Phosphorylation is Carried Out by the Src-Family Kinase Yes and Increases Under Chemokine Treatment.

In view of the presence of the putative SH3-binding motif close to the potential phosphorylatable site Y479, a possible interaction between CRMP2 and tyrosine kinases through its SH3 domain was studied. This was done using a membrane array bearing several protein SH3 domains that remain folded in active conformations. Ten different lymphocyte tyrosine kinases, including Abelson kinase (Abl), Src family kinases (Lck, Yes, c-Src, Fyn, Hck, Blk) and Tec family kinases (Itk, Txk, Btk) were present in this array. Following His-tagged recombinant CRMP2 hybridization, protein-protein interactions were visualized using anti-His antibody and spot intensities revealed the interaction strength (Table 1).

TABLE 1
Identification of SH3 protein-CRMP2 interaction

Strength of
interaction
with CRMP2

Tyrosine kinase proteins

| | | |
|---|---|---|
| Yes1 | Yamaguchi sarcoma virus oncogen homolog 1 | ++ |
| Abl | Abelson tyrosine kinase | + |
| BLK | B-lymphocyte specific protein tyrosine kinase | + |
| LCK | Human T-lymphocyte specific protein tyrosine | − |
| FYN | Proto-oncogen tyrosine protein kinase | − |
| BTK | Bruton tyrosin kinase | − |
| c-Src | Cellular rous sarcoma virus oncogen homolog 1 | − |
| Hck | Hemopoietic cell kinase | − |
| TXK | Tyrosine-protein kinase TXK | − |
| Itk | Interleukin-2-inducible T-cell kinase | − |

Non-kinase proteins

| | | |
|---|---|---|
| PLC-γ | Phospholipase C gamma 1 | +++ |
| VAV1 | Vav proto-oncogen SH2 domain 1 | ++ |
| PI3beta | Phosphoinositide-3-kinase p85 regulatory β | ++ |
| | subunit | |
| ITSN-D1 | Intersectin, SH3 domain #1 | + |

CRMP2-His recombinant protein was incubated with a membrane spotted in duplicate with SH3 domains of 38 proteins (TranSignal TM SH3 Domain array I - Panomics) according to manufacturer's instructions.
Anti-His antibody revealed the association of CRMP2 with multiple SH3 domains, including those of some tyrosine kinase proteins.
Spot intensities (− to +++) indicated the binding affinity of SH3 domains to the ligand CRMP2 and revealed Yes as a potent tyrosine kinase candidate for CRMP2.

Yes kinase displayed a strong binding to CRMP2, while Blk and Abl showed weak signals. In addition, four non-kinase protein SH3-domains belonging to Vav1, PLCγ, ITSN and PI3β also displayed strong binding to CRMP2. Interestingly, PLCγ and ITSN have previously been observed as binding partners for CRMP2 (Quinn et al. (2003) J. Neurosci. 23:2815-2823; Buttner et al. (2005) Biochemistry 44:6938-6947). The inventors then focused on Yes, the more potent tyrosine kinase candidate for CRMP2 phosphorylation.

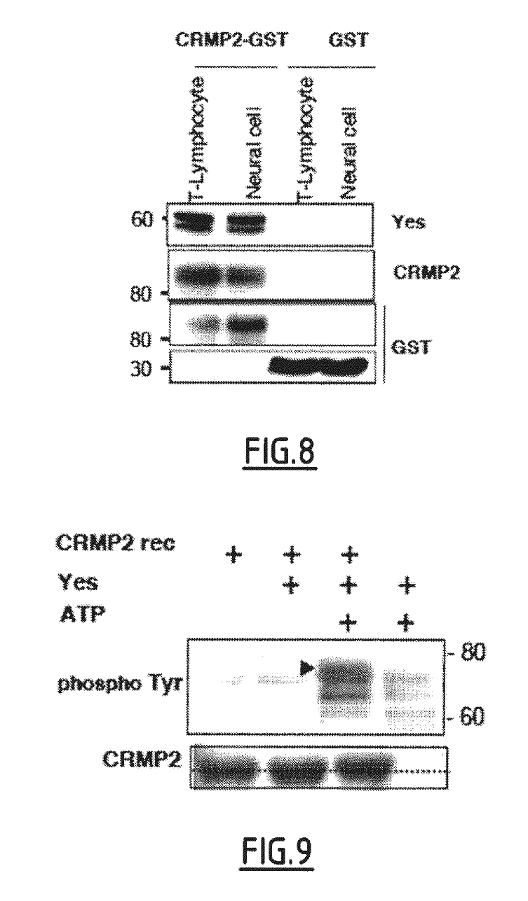

The Yes/CRMP2 interaction was evaluated by several approaches. First, localization of these proteins was assessed on primary T lymphocytes and Jurkat T-cells that had been allowed to adhere onto collagen-I coated coverslips and then treated with CXCL12 (100 ng/ml, 5 min). Immunofluorescence, performed with anti-Yes and anti-pep4 antibodies, showed the co-distribution of CRMP2 and Yes, especially at the uropod of polarized T-cells. Yes/CRMP2 interactions were next examined by a GST-pulldown assay using cell lysates from primary T-lymphocytes and from neural cells (Dev cell line) (FIG. 8), as CRMP2 is also involved in motility in the central nervous system (CNS). CRMP2 immobilized on glutathione-Sepharose beads was incubated with cell lysates. Western blots, performed on eluates from both cell types, showed the presence of Yes protein in association with CRMP2-GST, but not with GST alone. Taken together, these results defined the Yes kinase as a potent binding partner for CRMP2. In order to evaluate the functional significance of this interaction, an in vitro kinase assay was performed using active recombinant human Yes kinase and His-tagged CRMP2 as a substrate (FIG. 9). Phosphorylation was detected using an anti-phospho-Tyrosine antibody by immunoblotting. A control was carried out in the absence of CRMP2, which showed Yes self-phosphorylation. A band corresponding to CRMP2 phosphorylation was detected only in the presence of ATP. As a consequence of protein phosphorylation, this band displayed a slight increase in molecular weight.

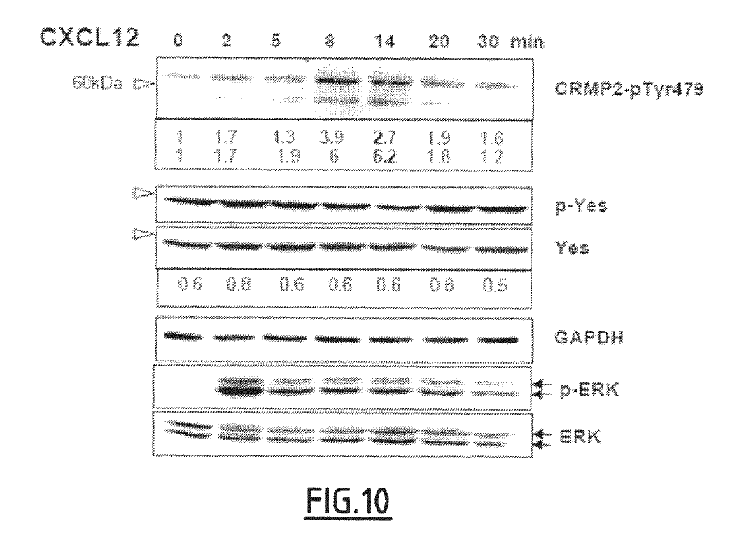

To confirm the presence of tyrosine-phosphorylated forms of CRMP2 in T-cells, a polyclonal antibody was raised against a fragment of the CRMP2 sequence (AA470-483) containing the phosphorylated residue Tyr479. Immunoblotting with this antibody revealed the presence of CRMP2-pTyr479 in T-lymphocytes, detected as both full-length and 58 kDa cleaved proteins. Examination of CRMP2-pTyr479 in Jurkat T-cells treated with CXCL12 (100 ng/ml; 0, 1, 2, 4, 6, 10, 15, 30 min) showed an increase in Tyr479 phosphorylation (FIG. 10), mainly observed at 8-14 min post-treatment. Increased expression of CRMP2-pTyr479 was concomitant with the decrease of CRMP2-Thr509/514 suggesting their dependence. This peak in Tyr479 phosphorylation correlated with the activation of Src-family kinases at this time point, as shown by immunoblotting using anti-Src-pTyr416, an antibody that recognizes phosphorylated Src family members, including Yes. Immunofluorescence was then performed on Jurkat cells treated with CXCL12 (15 min) using antibodies against the phosphorylated and non-phosphorylated CRMP2 forms. Staining for CRMP2-pTyr479 was mainly with a polarized distribution in T lymphocytes. Co-localization with vimentin showed that, compared to the CRMP2 forms recognized by the anti-pep4 and anti-C-ter antibodies, which were either associated or not associated with vimentin, respectively, the phosphorylated CRMP2-Tyr479 was mainly colocalized with vimentin at the trailing pole. Taken together, these results identified a new form of phosphorylated CRMP2 that was modulated by CXCL12 signaling, colocalized with cytoskeletal elements and could be targeted by the Src-family kinase Yes.

### CRMP2-Tyr479 Phosphorylation is Involved in Chemokine-Induced Polarization and Migration of T-Cells.

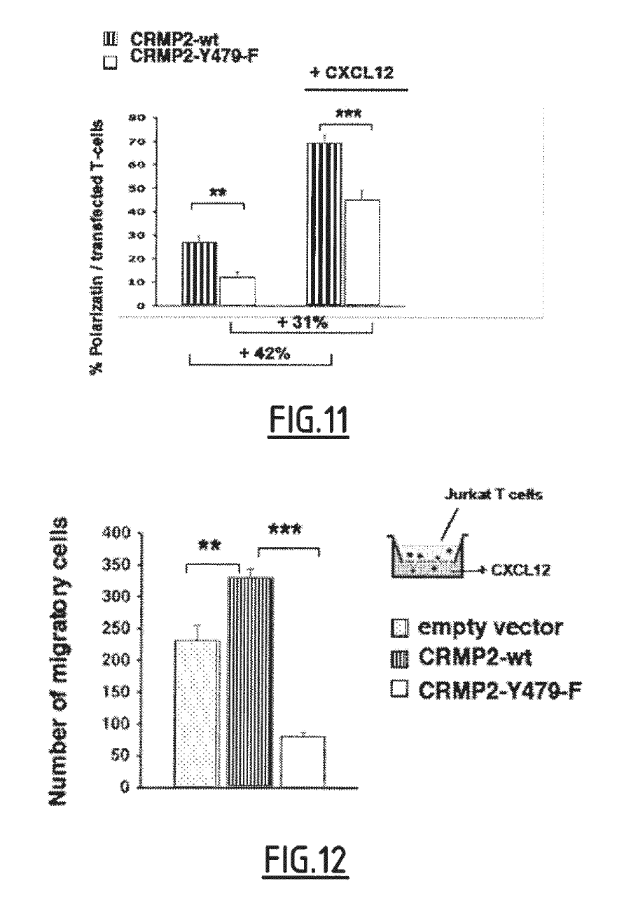

To assess the functional significance of CRMP2 phosphorylation on Tyr479, the inventors engineered the mutation Y479-F on the full-length CRMP2 sequence. The effect of Tyr479 phosphorylation impairment on T-cell polarization was then analyzed in Jurkat T-cells transiently transfected with Flag-tagged CRMP2-wt and CRMP2-Y479-F mutant. Twenty-four hours after transfection, T-cells were allowed to adhere onto collagen-1-coated slides, then treated with CXCL12 and examined by fluorescence microscopy. Polarization of CRMP2 in transfected T-cells, as visualized by Flag-positive immunostaining, was examined based on vimentin network co-localization. This allowed the inventors to evaluate the polarization of Flag positive T-cells transfected either with CRMP2-wt or CRMP2-Y479-F (expressed as a percentage of all transfected cells). The un-treated Jurkat T-cell population transfected with CRMP2-wt displayed ˜28% spontaneously polarized cells, but this clearly decreased in T-cells transfected with the CRMP2-Y479-F mutant (FIG. 11). Following CXCL12 treatment, vimentin was quickly redistributed to the uropod in CRMP2-wt transfected T-cells. In contrast, CRMP2-Y479-F transfected cells were clearly less polarized (FIG. 11). In addition, the increase in the number of polarized cells following CXCL12 treatment was lower in Jurkat T-cells transfected with CRMP2-Y479-F than with CRMP2-wt (31% versus 42%, respectively) (FIG. 11), thus confirming the impact of Tyr479 phosphorylation on T-cell polarization. These results clearly showed the role of CRMP2-Tyr479 phosphorylation in T lymphocyte polarization.

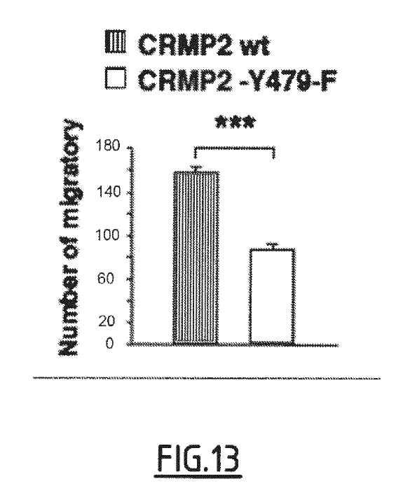

As T-cell polarization is a prerequisite for migration, the inventors further evaluated the influence of Tyr479 phosphorylation on T-cell migration. Thus, the inventors first assessed the ability of transfected Jurkat T-cells to migrate towards CXCL12, by performing a transmigration assay in Transwell chambers. As shown in FIG. 12, the rate of migration of CRMP2-Y479-F transfected cells was drastically reduced compared to those with CRMP2-wt and control cells (empty vector). Beyond T-cell transmigration that is necessary to traverse blood vessels, migration within invaded tissue is also a key point, especially within the CNS where CXCL12 and its cognate receptor are constitutively expressed. The inventors therefore examined whether Tyr479 phosphorylation had an influence on T-cell migration within neural tissue, using mouse hippocampal organotypic culture. Transfected Jurkat T-cells (40-50% transfection efficiency) were stained with the vital dye CFSE in order to easily visualize them both on and in neural tissue. Cells were then spotted close to brain slices and were counted after 18 hours incubation. CRMP2-Y479-F transfected cells displayed a reduced ability to travel on neural tissue compared to wild type transfected cells (FIG. 13). These results demonstrated the role of CRMP2-Tyr479 phosphorylation in the process of T-cell migration within neural tissue.

Accordingly the present inventors demonstrated that the phosphorylation on tyrosine 479 had an impact on the T cells migration, and accordingly was usable as a predictive marker of inflammatory diseases of the CNS.

### EXAMPLE 2

The present inventors have shown that the activation of T lymphocytes mediated by TCR stimulation led to an increase in the detection of CRMP2, more particularly of the cleaved form of CRMP2, by the anti-peptide 4 antibodies described in the international application WO2003/022298.

Interestingly, these antibodies strongly recognized column-enriched phosphorylated forms of CRMP2. Moreover, S465-phosphorylated CRMP2 is the main phosphorylated form of CRMP2 among phosphorylated forms of CRMP2 described in the CNS.

Accordingly, these results suggest that the detection of cleaved S465-phosphorylated CRMP2 is associated with the activation of T lymphocytes mediated by TCR stimulation, and can be used as a peripheral marker of TCR activation.

### EXAMPLE 3

The present inventors have shown that in patients suffering of multiple sclerosis or of myelopathy associated with an HTLV-1 infection, a subpopulation of activated T lymphocytes (CD69+ and/or HLA-DR+) expressed more strongly CRMP2 than in healthy subjects.

The inventors showed that this modification was associated with an increase in T lymphocytes migratory capacity. Moreover this increase can be inhibited using anti-CRMP2 antibodies.

The high expression of CRMP2 was detected using anti-peptide 4 antibodies. Since these antibodies recognize particularly a phosphorylated and cleaved form of CRMP2, this increased detection is probably due to a modification of CRMP2 phosphorylation, in particular to the S465 phosphorylation of CRMP2.

Accordingly, these results demonstrate that the cleavage of CRMP2 and the overexpression of phosphorylated CRMP2 in T lymphocytes (on serine 465 via TCR stimulation and on tyrosine 479 via chemokines activation) are peripheral markers of neuroinflammatory processes and can be used in early diagnostic, prognostic or monitoring of inflammatory diseases of the central nervous system.

1. A method for in vitro prognosis, diagnosis and/or monitoring of an inflammatory disease of the central nervous system in a subject, said method comprising detecting, in a sample of cells of the immune system from the subject, the presence of a Collapsin Response Mediator Protein 2 (CRMP2) which is phosphorylated on tyrosine 479 (Y479), wherein the detection of the presence of Y479-phosphorylated CRMP2 is indicative of an inflammatory disease of the central nervous system.

2. The method according to claim 1, wherein said Y479-phosphorylated CRMP2 is in a cleaved form.

3. The method according to claim 2, wherein said Y479-phosphorylated CRMP2 is further phosphorylated on serine 465 (S465).

4. The method according to claim 1, wherein said Y479-phosphorylated CRMP2 is detected with an antibody specific of for Y479-phosphorylated CRMP2.

5. The method according to claim 1, wherein said inflammatory disease of the central nervous system is selected from the group consisting of viral or bacterial infections with meningitis, encephalitis, myelitis, encephalomyelitis, encephalititis, myelopathy, multiple sclerosis, Parkinson disease and Alzheimer disease.

6. An antibody specific for a CRMP2 which is phosphorylated on tyrosine 479.

7. An antibody specific of for a CRMP2 which is phosphorylated on serine 465.

8-16. (canceled)

17. A method of decreasing immune cell migration in a subject, comprising the step of administering to the subject one or both of
i) an antibody specific for CRMP2 which is phosphorylated on tyrosine 479; and
ii) an antibody specific for CRMP2 which is phosphorylated on serine 465.

18. A method of treating an inflammatory disease of the central nervous system in a subject, comprising the step of
administering to the subject one or both of
i) an antibody specific for a CRMP2 which is phosphorylated on tyrosine 479; and
ii) an antibody specific for a CRMP2 which is phosphorylated on serine 465.

19. The method according to claim 18, wherein said inflammatory disease of the central nervous system is selected from the group consisting of viral or bacterial infections with meningitis, encephalitis, myelitis, encephalomyelitis, encephalititis, myelopathy, multiple sclerosis, Parkinson disease and Alzheimer disease.

20. A method of detecting a CRMP2 which is phosphorylated on tyrosine 479 and/or on serine 465, comprising the step of
contacting said CRMP2 with an antibody specific for a CRMP2 which is phosphorylated on tyrosine 479 and/or an antibody specific for a CRMP2 which is phosphorylated on serine 465.

21. The method according to claim 20, wherein said CRMP2 is in a cleaved form.

22. A method for decreasing migration of immune cells in a subject, comprising the step of
administering to said subject an antagonist of the CXCR4 receptor.

23. A method for treating an inflammatory disease of the central nervous system in a subject, comprising the step of
administering to said subject an antagonist of the CXCR4 receptor.

24. The method according to claim 23, wherein said inflammatory disease of the central nervous system is selected from the group consisting of viral or bacterial infections with meningitis, encephalitis, myelitis, encephalomyelitis, encephalititis or myelopathy, multiple sclerosis, Parkinson disease and Alzheimer disease.
